SEQUENCE LISTING (4 sequences)

SEQ ID NO: 1 - DNA, 120 bp, Artificial Sequence
  Synthetic (single strand DNA)
ccctatagtgagtcgtattacagcagatgtggatcagcaagcaggagtatgacgagtccggcccctccatcgtccaccgcaaatgttctaggcggactatgacttagttgcgttacaccc

SEQ ID NO: 2 - RNA, 20 nt, Artificial Sequence
  Synthetic (T7 promoter specific primer RNA)
uaauacgacucacuauaggg

SEQ ID NO: 3 - RNA, 24 nt, Artificial Sequence
  Synthetic (Non-specific primer RNA)
ccacccauggcaaauuccauggca

SEQ ID NO: 4 - RNA, 21 nt, Artificial Sequence
  Synthetic (Beta-actin specific primer RNA)
cagcagauguggaucagcaag

(19) United States
(12) Patent Application Publication
(10) Pub. No.: US 2014/0162276 A1
(43) Pub. Date: Jun. 12, 2014

(54) METHOD OF AMPLIFYING TARGET NUCLEIC ACID, METHOD OF ANALYZING TARGET NUCLEIC ACID, KIT FOR AMPLIFYING TARGET NUCLEIC ACID, AND COMPOSITION FOR AMPLIFYING TARGET NUCLEIC ACID

(71) Applicant: Samsung Electronics Co., Ltd., Suwon-si (KR)

(72) Inventors: Joo-won RHEE, Yongin-si (KR); Sea-hee KIM, Seoul (KR); Ko-bong CHOI, Osan-si (KR)

(73) Assignee: Samsung Electronics Co., Ltd., Suwon-si (KR)

(21) Appl. No.: 14/082,250

(22) Filed: Nov. 18, 2013

(30) Foreign Application Priority Data
Dec. 6, 2012 (KR) 10-2012-0141198

(51) Int. Cl. C12Q 1/68

(52) U.S. Cl. CPC C12Q 1/6844
USPC 435/6.12; 435/91.2

(57) ABSTRACT

Provided are methods of efficiently amplifying or analyzing a target nucleic acid and a kit and a composition to efficiently amplify a target nucleic acid.

### CROSS-REFERENCE TO RELATED APPLICATION

This application claims the benefit of Korean Patent Application No. 10-2012-0141198, filed on Dec. 6, 2012 in the Korean Intellectual Property Office, the entire disclosure of which is incorporated herein by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 1,310 Bytes ASCII (Text) file named “713927_ST25.TXT,” created on Nov. 15, 2013.

### BACKGROUND

1. Field

The present disclosure relates to methods of efficiently amplifying or analyzing target nucleic acids and kits and compositions for efficiently amplifying target nucleic acids.

2. Description of the Related Art

Methods of amplifying nucleic acids such as strand displacement amplification (SDA), rolling circle amplification (RCA), polymerase chain reaction (PCR), and nucleic acid sequence based amplification (NASBA) are already well known.

RCA driven by nucleic acid polymerase may amplify circular nucleic acids under isothermal conditions. When a single primer is used, RCA produces tandem sequences including target nucleic acid repeat units. DNA produced by RCA may be further labeled to be analyzed.

However, despite these well-known methods of amplifying target nucleic acids, methods that may efficiently amplify target nucleic acids are still required.

### SUMMARY

Provided are methods of efficiently amplifying target nucleic acids. In one aspect there is provided a method of amplifying a target nucleic acid, the method comprising: providing a circular single strand nucleic acid comprising a sequence identical to at least a portion of at least one strand of a target nucleic acid or a sequence complementary to at least a portion of at least one strand of a target nucleic acid, a first primer comprising a sequence complementary to the circular single strand nucleic acid, and a sample comprising a target nucleic acid; incubating the circular single strand nucleic acid, the first primer, and the sample wherein the primer hybridizes to the circular single strand nucleic acid; and incubating the hybridization product in the presence of nucleic acid polymerase to amplify the target nucleic acid. In another aspect, the method comprises: providing a circular single strand nucleic acid comprising a sequence complementary to at least one strand of a target nucleic acid, and a sample comprising the target nucleic acid; incubating the circular single strand nucleic acid and the sample to hybridize the circular single strand nucleic acid to the target nucleic acid; and incubating the hybridization product in the presence of nucleic acid polymerase to amplify the target nucleic acid.

Also provided are related methods, kits, and compositions.

### BRIEF DESCRIPTION OF THE DRAWINGS

These and/or other aspects will become apparent and more readily appreciated from the following description of the embodiments, taken in conjunction with the accompanying drawings in which:

FIG. 1 is a photograph illustrating a result of isothermal amplification using circular single-strand nucleic acid as a template and target RNA as a primer; and

FIG. 2 is a diagram illustrating an isothermal amplification result obtained by using circular single-strand nucleic acid as a template and a different copy number of target RNA as a primer.

### DETAILED DESCRIPTION

Reference will now be made in detail to embodiments, examples of which are illustrated in the accompanying drawings, wherein like reference numerals refer to the like elements throughout. In this regard, the present embodiments may have different forms and should not be construed as being limited to the descriptions set forth herein. Accordingly, the embodiments are merely described below, by referring to the figures, to explain aspects of the present description. Expressions such as “at least one of,” when preceding a list of elements, modify the entire list of elements and do not modify the individual elements of the list.

According to an aspect of the present invention concept, a method of amplifying target nucleic acids includes incubating circular single strand nucleic acids, a first primer having a sequence complementary to the circular single-strand nucleic acids, and a sample including target nucleic acids, whereupon the circular single strand nucleic acids hybridize with the first primer. The hybridization product may be incubated in the presence of nucleic acid polymerase to amplify the target nucleic acids included in the sample. More specifically, incubating the hybridization product with a polymerase produces a transcript of the circular nucleic acid. Since the circular nucleic acid contains a sequence that is identical to a portion of the target sequence, the polymerization product contains a sequence that is complementary to a portion of the target sequence, such that the target nucleic acid if present can bind to the nucleic acid and act as a primer for amplification of the transcript of the circular nucleic acid. As used herein, the term “transcript” refers to the newly polymerized product which is complementary to the template strand regardless whether it is DNA or RNA.

The method of amplifying target nucleic acids includes incubating circular single strand nucleic acids, a first primer having a sequence complementary to the circular single strand nucleic acids, and a sample including target nucleic acids, whereby the circular single strand nucleic acid hybridizes with the first primer.

The incubation may be performed under a condition that ensures hybridization of the circular single strand nucleic acids with the first primer. The condition may be, for example, a process of removing double-stranded structure, 3-dimensional structure, or a combination thereof by raising a temperature followed by a process of annealing the circular single strand nucleic acids and the target nucleic acids to be hybridized to each other by lowering the temperature. In addition, the incubation may be performed at a temperature less than 14° C. However, the hybridization of the circular single strand nucleic acids with the first primer occurs at dynamic equilibrium so that thermal cycling is not necessarily required. Accordingly, the incubation may be performed under an isothermal condition, that is, a condition that does not require thermal cycling. The incubation may be conducted at about optimum temperature of a nucleic acid polymerase.

The incubation may be performed in the presence of a suitable medium, for example, water or a buffer such as phosphate buffered saline (PBS). In addition, the incubation may be performed simultaneously or sequentially in the same reaction container used for incubating the hybridization product in the presence of nucleic acid polymerase. Also, the incubation in the presence of the suitable medium may be performed under the same condition as the incubation in the presence of nucleic acid polymerase.

The circular single strand nucleic acids may be DNA, RNA, LNA, or a combination thereof and may include a sequence identical to at least a portion of at least one strand of the target nucleic acids or a sequence complementary to at least a portion of at least one strand of a target nucleic acid. The sequence identical to at least a portion of at least one strand of the target nucleic acids may include at least two consecutive nucleotides (e.g., at least 5 or at least 10 nucleotides) from the 5′-terminal end of the one strand of the target nucleic acids (e.g., the circular nucleic acid may contain a sequence identical to at least two (e.g., at least 5 or at least 10) consecutive nucleic acids of the target nucleic acid). The circular single strand nucleic acids may include a sequence of the target nucleic acids or a sequence complementary to the target nucleic acids, the target nucleic acids to be amplified. The circular single strand nucleic acids may vary depending on a length of the target nucleic acids which are to be amplified. A length of the circular single strand nucleic acids is about 16 nt to about 1,000 nt, for example, about 16 nt to about 800 nt, about 16 nt to about 500 nt, about 16 nt to about 300 nt, about 16 nt to about 200 nt, about 16 nt to about 150 nt, about 16 nt to about 120 nt, about 20 nt to about 150 nt, about 20 nt to about 120 nt, about 20 nt to about 100 nt, about 20 nt to about 50 nt, about 30 nt to about 200 nt, about 30 nt to about 150 nt, about 30 nt to about 120 nt, about 30 nt to about 100 nt, or about 30 nt to about 50 nt. The sequence identical to at least a portion of at least one strand of the target nucleic acids or the sequence complementary to at least a portion of at least one strand of a target nucleic acid may have at least 2 nt, 5 nt, 10 nt, 15 nt, 20 nt, 5-50 nt, 5-30 nt, 5-20 nt, 10-50 nt, 10-30 nt, or 10-20 nt.

The target nucleic acids may be DNA, RNA, or a combination thereof, and may be single strand nucleic acids. The target nucleic acids may be amplified as whole or as a segment thereof. The segment may be at least 5 nt, 10 nt, 15 nt, or 20 nt. The target nucleic acids may contain a 3′-hydroxyl group used to take part in the nucleotide polymerization reaction at the 3′-end of the at least one strand of the target nucleic acids. The 3′-hydroxyl group may be introduced to the 3′-end for example, during the fragmentation. When the sample includes double strand nucleic acids or very long nucleic acid, a process of fragmenting the double strand nucleic acids or the very long nucleic acid may be further included. The fragmentation may be performed by physical or chemical methods. The fragmentation may be performed by, for example, ultrasonic irradiation or nuclease such as endonuclease or exonuclease. In addition, when the nucleic acids are double-stranded, a process of denaturing the double strand nucleic acids may be included to produce nucleic acids that include single strands. The length of the target nucleic acids may be at least about 16 nt, 20 nt, 30 nt, 50 nt, 100 nt, 500 nt, or 100 nt. A length of the target nucleic acids may be about 16 nt to about 1,000 nt, for example, about 16 nt to about 800 nt, about 16 nt to about 500 nt, about 16 nt to about 300 nt, about 16 nt to about 200 nt, about 16 nt to about 150 nt, about 16 nt to about 120 nt, about 16 nt to about 30 nt, about 16 nt to about 25 nt, about 16 nt to about 20 nt, about 20 nt to about 150 nt, about 20 nt to about 120 nt, about 20 nt to about 100 nt, about 20 nt to about 50 nt, about 20 nt to about 30 nt, about 20 nt to about 25 nt, about 21 nt to about 25 nt, about 30 nt to about 200 nt, about 30 nt to about 150 nt, about 30 nt to about 120 nt, about 30 nt to about 100 nt, or about 30 nt to about 50 nt.

The sample may be any sample including the target nucleic acids. For example, the sample may be a biological sample such as blood, urine, saliva, tears, tissue section, or a combination thereof. The sample may include RNA separated from a biological sample. For example, the sample may include RNA, which is separated by a method of RNA separation, from a biological sample. Examples of the method of RNA separation are phenol-chloroform extraction, purification based on solid-phase such silica, or a combination thereof. The RNA may be mRNA, miRNA, tRNA, rRNA, or a combination thereof.

A first primer includes polynucleotide having a single strand region that may serve as a starting point for template-instructed DNA synthesis. The first primer may be DNA, RNA, or a combination thereof. The first primer may include about 10 nt to about 100 nt, for example, about 10 nt to about 80 nt, about 10 nt to about 60 nt, about 10 nt to about 40 nt, about 10 nt to about 30 nt, about 15 nt to about 100 nt, about 15 nt to about 80 nt, about 15 nt to about 60 nt, about 15 nt to about 40 nt, or about 15 nt to about 30 nt.

The method of amplifying target nucleic acids includes a process of incubating the hybridization product in the presence of nucleic acid polymerase to amplify the target nucleic acid. More specifically, incubating the hybridization product with a polymerase produces a transcript of the circular nucleic acid. Since the circular nucleic acid contains a sequence that is identical to a portion of the target sequence, the polymerization product contains a sequence that is complementary to a portion of the target sequence, such that the target nucleic acid if present can bind to the nucleic acid and act as a primer for amplification of the transcript of the circular nucleic acid.

The incubation may be performed under conditions that catalyze strand displacement replication of the circular single strand nucleic acids. For example, the incubation may be performed under conditions to catalyze rolling circle amplification replication. The conditions to catalyze replication of strand displacement may include an enzyme, a suitable temperature in the presence of a reagent, and a pH. The enzyme may be strand displacement nucleic acid polymerase. The reagent may be primer, dNTP, NTP, cofactor of strand displacement nucleic acid polymerase, buffer, or a combination thereof. The temperature may be about 20° C. to about 70° C., for example, about 25° C. to about 70° C., about 30° C. to about 70° C., about 35° C. to about 70° C., about 40° C. to about 70° C., about 25° C. to about 45° C., about 30° C. to about 45° C., about 35° C. to about 45° C., or about 40° C. to about 45° C. The incubation may be performed under isothermal condition. The term “isothermal” used in the present specification means that thermal cycling is not required, and does not necessarily indicate the fixed temperature condition. The incubation may cause nucleotide extension from the 3′-terminal of the first primer, which is hybridized with the circular single strand nucleic acids, to produce a transcript of the circular nucleic acid. In addition, the extension may induce a formation of tandem sequence DNA due to rolling circle amplification (RCA). Since the circular nucleic acid contains a sequence identical to a portion of the target sequence, the transcript or tandem sequence DNA contains a sequence that is complementary to the target sequence. Thus, when a target nucleic acid exists in the sample, it acts as a primer complementary to the tandem sequence and the tandem sequence DNA is further replicated and amplified.

The target nucleic acids may be extended by hybridizing with the tandem sequence extended from the first primer. Accordingly, multiple displacement amplification of the circular single strand nucleic acids may be performed. Each tandem sequence DNA includes multiple tandem repeat units of the same sequence. As a result, the target nucleic acids may be amplified.

The nucleic acid polymerase may be RNA-dependent DNA polymerase, DNA-dependent DNA polymerase, DNA-dependent RNA polymerase, or a combination thereof. The nucleic acid polymerase may include strand displacement polymerase activity. Examples of the nucleic acid polymerase including strand displacement polymerase activity are Bst DNA polymerse, exonuclease minus, Tth DNA polymerase, pyrophage (PYROPHAGE™) 3173 DNA polymerase, BcaBEST DNA polymerase, φ29 DNA polymerase, or a combination thereof. Examples of the DNA-dependent RNA polymerase are T7 RNA polymerase, T3 RNA polymerase, SP6 RNA polymerase, or a combination thereof.

The incubation of the hybridization product in the presence of nucleic acid polymerase may be performed in the presence of nucleoside triphosphates (NTP) or deoxynucleoside triphosphates (dNTP) including a detectable marker. The detectable marker may be a chromophore, an enzyme, or a ligand, and the chromophore may be a fluorophore. Examples of the detectable marker are 4′-6-diamidino-2-phenylindole(DAPI), fluorescein isothiocyanate (FITC), cyanine dyes Cy3, Cy3.5, Cy5, Cy5.5, or Cy7, or a combination thereof.

The incubation may or may not include a process of circularizing the target nucleic acids.

According to another aspect of the present invention concept, a method of analyzing target nucleic acids in a sample includes incubating circular single strand nucleic acids, a first primer having a sequence complementary to the circular single stranded nucleic acids, and a sample including target nucleic acids to hybridize the circular single strand nucleic acids with the first primer; incubating the hybridization product in the presence of nucleic acid polymerase to amplify the target nucleic acids; and measuring the amplified product to analyze the target nucleic acid in a sample.

The incubation of circular single strand nucleic acids, a first primer having a sequence complementary to the circular single strand nucleic acids, and a sample including target nucleic acids to hybridize the circular single strand nucleic acids with the first primer; and the incubation of the hybridization product in the presence of nucleic acid polymerase to amplify the target nucleic acids are the same as described above.

The method of analyzing target nucleic acids includes measuring the amplified product to analyze the target nucleic acid in a sample. The measuring of the amplified product may be performed by methods that are known to one of ordinary skills in the art. For example, the amplified product may be measured by staining dyes such as EtBr thereto after electrophoresis. The measurement may be performed by electrical or chemical methods. A chemical method includes an analysis of sequence on the extended product and a confirmation of a sequence produced by the hybridization. Also, a method of detecting a detectable marker labeled on the extended product may be included in the method of analyzing target nucleic acids.

The method of analyzing target nucleic acids may include a process of correlating the amplified product with the target nucleic acids. The amplified product targets may be obtained from strand displacement amplification (SDA) or rolling circle amplification (RCA) and be concatemers that include at least one target nucleic acid. Thus, despite a low copy number of the target nucleic acids in the sample, the target nucleic acids are extended by SDA with the circular single strand nucleic acid as a template. Accordingly, the extension may be performed theoretically indefinitely so that a low copy number of the target nucleic acids also may be efficiently amplified. Furthermore, the extension may be performed by multiple displacement amplification (MDA), resulting in more efficient amplification. Accordingly, the presence or amount of the amplified product may actually be considered as the presence or amount of the target nucleic acids.

The method of analyzing target nucleic acids may include determining the presence of the target nucleic acids when the amplified product exists. Also, the method of analyzing target nucleic acids may include determining the amount of the target nucleic acids based on the amount of the amplified product.

In the method of analyzing target nucleic acids, incubating the circular single strand nucleic acids and the sample including the target nucleic acid may be performed simultaneously or sequentially with a following process of incubating the hybridization product in the presence of nucleic acid polymerase.

According to another aspect of the present invention concept, a method of amplifying target nucleic acids includes incubating circular single strand nucleic acids and a sample including target nucleic acids to hybridize the circular single strand nucleic acids with the target nucleic acids, wherein the circular single strand nucleic acids include a sequence complementary to the target nucleic acids; and incubating the hybridization product in the presence of nucleic acid polymerase to amplify the target nucleic acids.

The method of amplifying target nucleic acids includes incubating circular single strand nucleic acids and a sample including the same to hybridize the circular single strand nucleic acids with the target nucleic acid, wherein the circular single strand nucleic acids include a sequence complementary to the target nucleic acids.

The incubation of the circular single strand nucleic acids and the sample including the same may be performed under a condition to hybridize the circular single strand nucleic acids with the target nucleic acids. The condition used in the present specification may include, for example, a process of removing double-stranded structure, 3-dimensional structure, or a combination thereof by raising a temperature followed by a process of annealing the circular single strand nucleic acids and target nucleic acids to be hybridized each other by lowering the temperature. In addition, the incubation may be performed at a temperature less than 14° C. However, the hybridization of the circular single strand nucleic acids with the first primer occurs in dynamic equilibrium so that thermal cycling is not necessarily required. Accordingly, the incubation may be performed under an isothermal condition, that is, a condition that does not require thermal cycling. The incubation may be performed in the presence of suitable medium, for example, water or buffer such as PBS. The incubation may be conducted at about optimum temperature of a nucleic acid polymerase.

In addition, the incubation may be performed simultaneously or sequentially in the same reaction container used when incubating the hybridization product in the presence of nucleic acid polymerase. Also, the incubation in the presence of a suitable medium may be performed under the same condition as the incubation in the presence of nucleic acid polymerase.

The circular single strand nucleic acids may be DNA, RNA, LNA, or a combination thereof and may include a sequence complementary to a complementary strand of the target nucleic acids. The circular single strand nucleic acids may include a sequence complementary to at least one consecutive nucleotide from the 3′-terminal of the complementary strand of the target nucleic acids. The circular single strand nucleic acids may vary depending on a length of the target nucleic acids which are to be amplified. The length of the circular single strand nucleic acids may be at least about 16 nt, 20 nt, 30 nt, 50 nt, 100 nt, 500 nt, or 100 nt. A length of the circular single strand nucleic acids may be about 16 nt to about 1,000 nt, for example, about 16 nt to about 800 nt, about 16 nt to about 500 nt, about 16 nt to about 300 nt, about 16 nt to about 200 nt, about 16 nt to about 150 nt, about 16 nt to about 120 nt, about 20 nt to about 150 nt, about 20 nt to about 120 nt, about 20 nt to about 100 nt, about 20 nt to about 50 nt, about 30 nt to about 200 nt, about 30 nt to about 150 nt, about 30 nt to about 120 nt, about 30 nt to about 100 nt, or about 30 nt to about 50 nt.

The target nucleic acids may be DNA, RNA, or a combination thereof and may be single strand nucleic acids. The target nucleic acids may be amplified as a whole or as a segment thereof. The segment may be at least 5 nt, 10 nt, 15 nt, or 20 nt. The target nucleic acids may contain 3′-hydroxyl group used to take part in the nucleotide polymerization reaction at the 3′-end of the at least one strand of the target nucleic acids. The 3′-hydroxyl group may be introduced to the 3′-end for example, during the fragmentation. When the sample includes double strand nucleic acids or a very long nucleic acid, a process of fragmenting the double strand nucleic acids or the very long nucleic acid may be further included. The fragmentation may be performed by physical or chemical methods. The fragmentation may be performed by, for example, ultrasonic irradiation or nuclease such as endonuclease or exonuclease. In addition, when the nucleic acids are double-stranded, a process of denaturing the double strand nucleic acids may be performed to produce nucleic acids that include single strands. The length of the target nucleic acids may be at least about 16 nt, 20 nt, 30 nt, 50 nt, 100 nt, 500 nt, or 100 nt. A length of the target nucleic acids may be about 16 nt to about 1,000 nt, for example, about 16 nt to about 800 nt, about 16 nt to about 500 nt, about 16 nt to about 300 nt, about 16 nt to about 200 nt, about 16 nt to about 150 nt, about 16 nt to about 120 nt, about 16 nt to about 30 nt, about 16 nt to about 25 nt, about 16 nt to about 20 nt, about 20 nt to about 150 nt, about 20 nt to about 120 nt, about 20 nt to about 100 nt, about 20 nt to about 50 nt, about 20 nt to about 30 nt, about 20 nt to about 25 nt, about 21 nt to about 25 nt, about 30 nt to about 200 nt, about 30 nt to about 150 nt, about 30 nt to about 120 nt, about 30 nt to about 100 nt, or about 30 nt to about 50 nt.

The sample may be any of those including the target nucleic acids. For example, the sample may be a biological sample such as blood, urine, saliva, tears, tissue section, or a combination thereof. The sample may include RNA separated from a biological sample. For example, the sample may include RNA, which is separated by a method of RNA separation, from a biological sample. Examples of the method of RNA separation are phenol-chloroform extraction, purification based on solid-phase such silica, or a combination thereof. The RNA may be mRNA, miRNA, tRNA, rRNA, or a combination thereof.

The method of amplifying target nucleic acids includes incubating the hybridization product in the presence of nucleic acid polymerase to amplify the target nucleic acids.

The incubating may be performed under conditions to catalyze strand displacement replication of the circular single strand nucleic acids. For example, the incubation may be performed under conditions to catalyze rolling circle amplification replication. The conditions to catalyze replication of strand displacement may include an enzyme, a suitable temperature in the presence of a reagent, and a pH. The enzyme may be strand displacement nucleic acid polymerase. The reagent may be primer, dNTP, NTP, cofactor of strand displacement nucleic acid polymerase, buffer, or a combination thereof. The temperature may be about 20° C. to about 70° C., for example, about 25° C. to about 70° C., about 30° C. to about 70° C., about 35° C. to about 70° C., about 40° C. to about 70° C., about 25° C. to about 45° C., about 30° C. to about 45° C., about 35 to about 45° C., or about 40° C. to about 45° C. The incubation may be performed under an isothermal condition. The term “isothermal” used in the present specification means that thermal cycling is not required, and does not necessarily indicate the fixed temperature condition. The incubation may cause nucleotide extension from the 3′-terminal of the first primer, which is hybridized with the circular single strand nucleic acids. In addition, the extension may induce a formation of tandem sequence DNA due to rolling circle amplification (RCA). When a primer complementary to the tandem sequence DNA, i.e., target nucleic acids, exists, the tandem sequence DNA is replicated to form double strand tandem sequence DNA. Accordingly, the double stranded tandem sequence DNA may be formed. Each tandem sequence DNA includes multiple tandem repeat units of the same sequence. As a result, the target nucleic acids may be amplified.

The nucleic acid polymerase may be RNA-dependent DNA polymerase, DNA-dependent DNA polymerase, DNA-dependent RNA polymerase, or a combination thereof. The nucleic acid polymerase may include strand displacement polymerase activity. Examples of the nucleic acid polymerase including strand displacement polymerase activity are Bst DNA polymerse, exonuclease minus, Tth DNA polymerase, pyrophage (PYROPHAGE™) 3173 DNA polymerase, BcaBEST DNA polymerase, φ29 DNA polymerase, or a combination thereof. Examples of the DNA-dependent RNA polymerase are T7 RNA polymerase, T3 RNA polymerase, SP6 RNA polymerase, or a combination thereof.

The incubation of the hybridization product in the presence of nucleic acid polymerase may be performed in the presence of a second primer including a sequence of the circular single strand nucleic acid when the circular single strand nucleic acid has a sequence complementary to the target nucleic acids. The target nucleic acids may be single stranded nucleic acids. The second primer may hybridize to the transcript of the circular single strand nucleic acid. Due to the second primer, multiple displacement amplification (MDA) may be performed. The second primer may be DNA, RNA, LNA, or a combination thereof, and includes polynucleotide having a single strand region that may serve as a starting point for template-instructed DNA synthesis. The second primer may include about 10 nt to about 100 nt, for example, about 10 nt to about 80 nt, 10 nt to about 60 nt, 10 nt to about 40 nt, 10 nt to about 30 nt, 15 nt to about 100 nt, 15 nt to about 80 nt, 15 nt to about 60 nt, 15 nt to about 40 nt, or about 15 nt to about 30 nt.

The incubation of the hybridization product in the presence of nucleic acid polymerase may be performed in the presence of nucleoside triphosphates (NTP) or deoxynucleoside triphosphates (dNTP) including a detectable marker. The detectable marker may be a chromophore, an enzyme, or a ligand, and the chromophore may be a fluorophore. Examples of the detectable marker are 4′-6-diamidino-2-phenylindole(DAPI), fluorescein isothiocyanate (FITC), cyanine dyes Cy3, Cy3.5, Cy5, Cy5.5, or Cy7, or a combination thereof.

The incubation may or may not include circularizing the target nucleic acids.

According to another aspect of the present invention concept, a method of analyzing target nucleic acids in a sample includes incubating circular single strand nucleic acids and a sample including target nucleic acids to hybridize the circular single strand nucleic acid with the target nucleic acid, wherein the circular single strand nucleic acids include a sequence complementary to the target nucleic acids; incubating the hybridization product in the presence of nucleic acid polymerase to amplify the target nucleic acids; and measuring the amplified product to analyze the target nucleic acid in a sample.

The incubation of circular single strand nucleic acids and a sample including target nucleic acids to hybridize the circular single strand nucleic acids with the target nucleic acids, wherein the circular single strand nucleic acids include a sequence complementary to the target nucleic acids; and the incubation of the hybridization product in the presence of nucleic acid polymerase to amplify the target nucleic acids are the same as described above.

The method of analyzing target nucleic acids includes measuring the amplified product to analyze the target nucleic acid in a sample. The measuring of the amplified product may be performed by methods that are known to one of ordinary skills in the art. For example, the amplified product may be measured by staining dyes such as EtBr thereto after electrophoresis. The measuring may be performed by electrical or chemical methods. A chemical method includes an analysis of sequence on the extended product and a confirmation of a sequence produced by the hybridization. Also, a method of detecting a detectable marker labeled on the extended product may be included.

The method of analyzing target nucleic acids may include correlating the amplified product with the target nucleic acids. The amplified product targets may be resulted from strand displacement amplification (SDA) or rolling circle amplification (RCA) and be concatemers that include at least one target nucleic acid. Thus, despite a low copy number of the target nucleic acids in the sample, the target nucleic acids are extended by SDA with the circular single strand nucleic acid as a template. Accordingly, the extension may be performed theoretically indefinitely so that a low copy number of the target nucleic acids also may be efficiently amplified. Furthermore, when the second primer exists, the extension may be performed by MDA, resulting in more efficient amplification. Accordingly, the presence or amount of the amplified product may actually be considered as the presence or amount of the target nucleic acids.

The method of analyzing target nucleic acids may include determining the presence of the target nucleic acids when the amplified product exists. Also, the method of analyzing target nucleic acids may include determining the amount of the target nucleic acids based on the amount of the amplified product.

In the method of analyzing target nucleic acids, incubating the circular single strand nucleic acids and the sample including the target nucleic acid may be performed simultaneously or sequentially with a following process of incubating the hybridization product in the presence of nucleic acid polymerase.

According to another aspect of the present invention concept, a kit for amplifying target nucleic acids includes circular single strand nucleic acid having a sequence complementary to target nucleic acids or to a complementary strand of the target nucleic acids; and nucleic acid polymerase.

The circular single strand nucleic acids may be DNA, RNA, LNA, or a combination thereof and may include a sequence complementary to at least one consecutive nucleotide from the 3′-terminal of the target nucleic acids or of the complementary strand of the target nucleic acids. The circular single strand nucleic acids may vary depending on a length of the target nucleic acids which are to be amplified. A length of the circular single strand nucleic acids is about 16 nt to about 1,000 nt, for example, about 16 nt to about 800 nt, about 16 nt to about 500 nt, about 16 nt to about 300 nt, about 16 nt to about 200 nt, about 16 nt to about 150 nt, about 16 nt to about 120 nt, about 20 nt to about 150 nt, about 20 nt to about 120 nt, about 20 nt to about 100 nt, about 20 nt to about 50 nt, about 30 nt to about 200 nt, about 30 nt to about 150 nt, about 30 nt to about 120 nt, about 30 nt to about 100 nt, or about 30 nt to about 50 nt.

The target nucleic acids may be DNA, RNA, or a combination thereof and may be single strand nucleic acids. In addition, the target nucleic acid may be present in a sample, and the sample may be any of those including the target nucleic acids. For example, the sample may be a biological sample such as blood, urine, saliva, tears, tissue section, or a combination thereof. The sample may include RNA separated from a biological sample. For example, the sample may include RNA, which is separated by a method of RNA separation, from a biological sample. Examples of the method of RNA separation are phenol-chloroform extraction, purification based on solid-phase such silica, or a combination thereof. The RNA may be mRNA, miRNA, tRNA, rRNA, or a combination thereof.

The nucleic acid polymerase may be RNA-dependent DNA polymerase, DNA-dependent DNA polymerase, DNA-dependent RNA polymerase, or a combination thereof. The nucleic acid polymerase may include strand displacement polymerase activity. Examples of the nucleic acid polymerase including strand displacement polymerase activity are Bst DNA polymerse, exonuclease minus, Tth DNA polymerase, pyrophage (PYROPHAGE™) 3173 DNA polymerase, BcaBEST DNA polymerase, φ29 DNA polymerase, or a combination thereof. Examples of the DNA-dependent RNA polymerase are T7 RNA

The kit may further include a description to be used to amplify the target nucleic acids.

The kit may further include a primer having a sequence complementary to the circular single strand nucleic acids or to a complementary sequence of the circular single strand nucleic acids.

According to another aspect of the present invention concept, a composition for amplifying target nucleic acids includes circular single strand nucleic acids having a sequence complementary to the target nucleic acids or to a complementary sequence of the target nucleic acids. The circular single strand nucleic acids are the same as described above.

One or more embodiments of the present invention concept will now be described in detail with reference to the following examples. However, these examples are not intended to limit the scope of the one or more embodiments of the present invention.

### Example 1

### Detection of Target Nucleic Acids Using Circular Single Strand Nucleic Acids as Templates

In the present example, the presence of target nucleic acids was confirmed by amplification using circular single strand nucleic acids and target nucleic acids as a template and a primer, respectively.

(1) Amplification of Target Nucleic Acids Using Circular Single Strand Nucleic Acids as Templates

A single strand DNA with 120 nucleotides of SEQ ID. NO: 1 was synthesized as circular single strand nucleic acids. The synthesized single strand DNA included a sequence, that is, a sequence from the first to 20^{th} nucleotide of SEQ ID. NO: 1, which is complementary to 20 nucleotides of a T7 promoter nucleotide sequence ( ) and a sequence that is, from the 21^{st} to about 120^{th} nucleotides of SEQ ID. NO: 1, which is complementary to 100 nucleotides of a β-actin gene nucleotide sequence.

The circular DNA was prepared from a synthesized single-stand DNA, which had 5′-phosphate and 3′-OH. The single-strand circular nucleic acid was prepared by incubating about 100 ng of the synthesized single-strand DNA at 60° C. for an hour in a reaction mixture including 1 unit CIRCLIGASE™ II (Epicenter, Cat. No. CL9021K) into a reaction buffer (33 mM Tris-acetate (pH 7.5), 66 mM potassium acetate, 0.5 mM DTT) and proceeding to self-ligation.

Next, a reaction mixture where about 0.5 ng of circular single-strand nucleic acid, 0.5 μM of each primer, and 1 unit Bst DNA polymerase (NEB, Cat. No. MO075L) were added into 1×MDA reaction buffer (20 mM Tris-HCl, 10 mM (NH₄)₂SO₄,10 mM KCl, 2 mM MgSO₄, 0.1% Triton X-100, pH 8.8 @ 25° C.) (Nanohelix) was incubated at a temperature of 65° C. for an hour. The primer combinations used were as follows: (a) 0.5 μM of T7 promoter primer RNA (SEQ ID. NO: 2) with a sequence identical to nucleotides from the first to the 20^{th} position in SEQ ID. NO: 1; (b) 0.5 μM of a T7 promoter primer RNA (SEQ ID. NO: 2) and 0.5 μM of a nonspecific primer RNA (SEQ ID. NO: 3) that is not complementary to the circular single-stranded nucleic acid; and (c) and 0.5 μM of a T7 promoter primer RNA (SEQ ID. NO: 2) and 0.5 μM of RNA (SEQ ID. NO: 4; RNA sequence corresponding to nucleotides from the 21^{st} to about 120^{th} positions in SEQ ID. NO: 1) corresponding to the nucleotide sequence of the β-actin gene of the circular single-stranded nucleic acid, respectively. All of the primers used herein are RNAs.

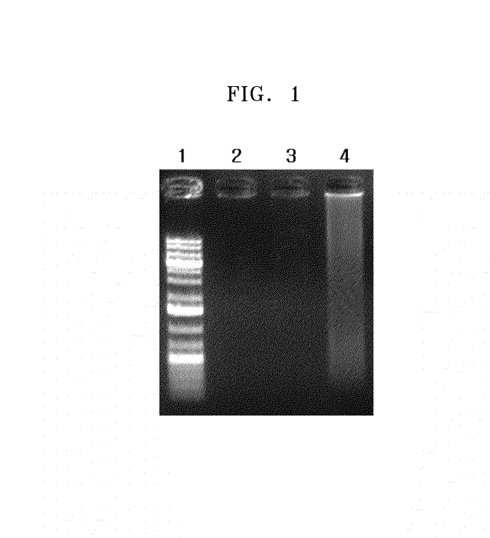

FIG. 1 is a photograph illustrating a result of isothermal amplification using circular single-stranded nucleic acid as a template and target RNA as a primer. In FIG. 1, lane 1 represents a size marker, while other lanes represent results of the electrophoresis of the products obtained by using the above-mentioned primers. For example, lane 2 represents a result using 0.5 μM of a T7 promoter primer RNA (SEQ ID. NO: 2); lane 3 represents results using 0.5 μM of a T7 promoter primer RNA (SEQ ID. NO: 2) and 0.5 μM of a nonspecific RNA (SEQ ID. NO: 3), which is not complementary to the circular single-strand nucleic acid, and lane 4 represents results using 0.5 μM of a T7 promoter primer RNA (SEQ ID. NO: 2) and 0.5 μM of a primer RNA (SEQ ID. NO: 4), which is specific to the β-actin gene.

As illustrated in FIG. 1, when 0.5 μM of a T7 promoter primer RNA (SEQ ID. NO: 2) was used (see lane 2), the target nucleic acid was not amplified. The same result was obtained when 0.5 μM of a T7 promoter primer RNA (SEQ ID. NO: 2) and 0.5 μM of a nonspecific primer RNA (SEQ ID. NO: 3) which is not complementary to the circular single-stranded nucleic acid were used (see lane 3). Based on the result of lane 4 in FIG. 1, existence of a T7 promoter primer sequence or a primer sequence specific to β-actin gene was confirmed. Herein, when a primer added from outside into the reaction mixture is a T7 promoter primer RNA (hereinafter, referred to “forward primer addition type”), existence of the sequence specific to the β-actin gene may be confirmed. Meanwhile, when a primer added from outside into the reaction mixture is a primer RNA specific to a β-actin gene (hereinafter, referred to “reverse primer addition type”), existence of the T7 promoter primer sequence may be confirmed. In addition, when no primer is added from the outside (hereinafter, referred to “no primer addition type”), it may be confirmed that both primer sequence specific to the β-actin gene and T7 promoter primer sequence are present in a sample. Also, based on the standard experimental result obtained experiment using samples having different amounts of target nucleic acids, the amount of target nucleic acids may be confirmed.

(2) Amplification Efficiency According to Concentrations of Target Nucleic Acids

A reaction mixture to which about 0.5 ng of circular single-strand nucleic acid as described in (1), 0.5 μM of T7 promoter primer RNA (SEQ ID. NO: 2), RNA (RNA sequence corresponding to nucleotides from the 21^{st} to the 41^{st} position in SEQ ID. NO: 1: SEQ ID NO: 4) corresponding to nucleotide sequences of the β-actin gene in other circular single-strand nucleic acid having different copy numbers (3×10², 3×10⁵, 3×10⁸, and 3×10¹¹ copies), and 1 unit Bst DNA polymerase (NEB, Cat. No. MO075L) were added into 1×MDA reaction buffer (20 mM Tris-HCl, 10 mM (NH₄)₂SO₄,10 mM KCl, 2 mM MgSO₄, 0.1% Triton X-100, pH 8.8 @ 25° C.) (Nanohelix) was incubated at a temperature of 65° C. for about one and a half hours. As a control group, the presence of circular single-stranded nucleic acid and no presence of a primer, or no presence of the RNA (SEQ ID. NO: 4) specific to the β-actin gene and T7 promoter primer RNA (SEQ ID. NO: 2) only was used as a primer.

Then, the fluorescence intensity according to the time of the amplification reaction was measured by using LC480 (Roche, lightcycler LC480) in regard with the reaction mixture. The detection reagent was 1× picogreen of QUANT-IT™ PICOGREEN™ dsDNA reagent (P7589, Invitrogen).

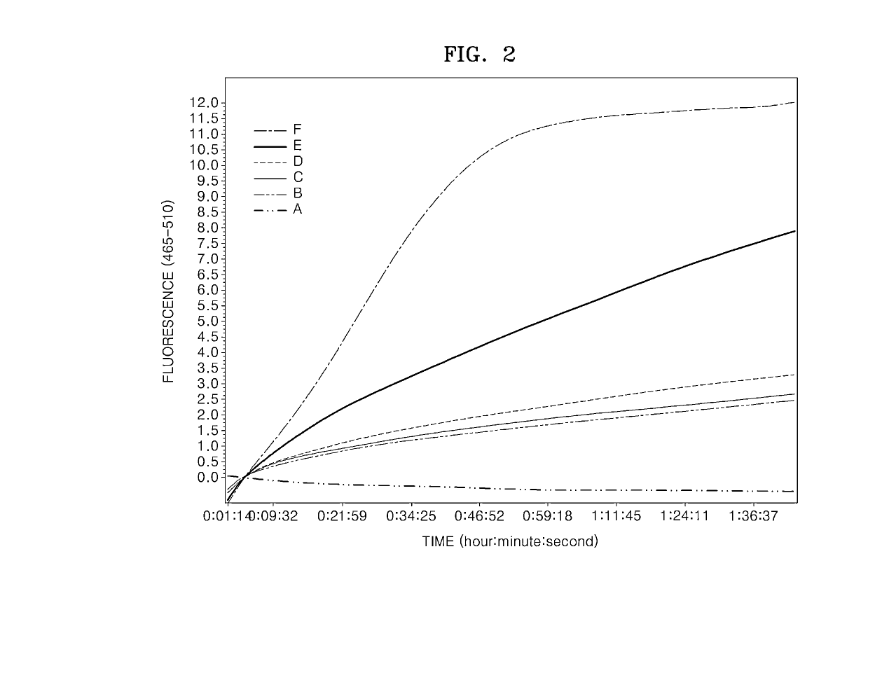

FIG. 2 is a diagram illustrating an isothermal amplification result using circular single-strand nucleic acid as a template and a different copy number of target RNA as a primer. In FIG. 2, A is a result obtained in the presence of the circular single-stranded nucleic acid and in the absence of the primer, while B is a result obtained by using a T7 promoter primer RNA (SEQ ID. NO: 2) only as a primer instead of a β-actin gene specific RNA (SEQ ID. NO: 4) RNA. C, D, E, and F are results obtained when a number of β-actin gene specific RNA (SEQ ID. NO: 4) copy is 3×10², 3×10⁵, 3×10⁸, and 3×10¹¹, respectively. As illustrated in FIG. 2, a target RNA of 3×10², 3×10⁵, 3×10⁸, and 3×10¹¹ copies has been sufficiently amplified. According to the method of the present invention, a target nucleic acid may be amplified with a high sensitivity, linearity, and/or a dynamic range.

According to an aspect of the present invention concept, target nucleic acids may be efficiently amplified.

According to another aspect of the present invention concept, target nucleic acids may be efficiently analyzed.

According to another aspect of the present invention concept, a kit may be used to amplify target nucleic acids.

According to another aspect of the present invention concept, a composition may be used to efficiently amplify target nucleic acids.

It should be understood that the exemplary embodiments described therein should be considered in a descriptive sense only and not for purposes of limitation. Descriptions of features or aspects within each embodiment should typically be considered as available for other similar features or aspects in other embodiments.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of amplifying a target nucleic acid, the method comprising:
providing a circular single strand nucleic acid comprising a sequence identical to at least a portion of at least one strand of a target nucleic acid or a sequence complementary to at least a portion of at least one strand of a target nucleic acid, a first primer comprising a sequence complementary to the circular single strand nucleic acid, and a sample comprising a target nucleic acid;
incubating the circular single strand nucleic acid, the first primer, and the sample wherein the primer hybridizes to the circular single strand nucleic acid;
incubating the hybridization product in the presence of nucleic acid polymerase to amplify the target nucleic acid.

2. The method of claim 1, wherein the circular single stranded nucleic acid is DNA, RNA, LNA, or a combination thereof.

3. The method of claim 1, wherein the circular single strand nucleic acid is about 16 nucleotides (nt) to about 1,000 nt.

4. The method of claim 1, wherein the target nucleic acid is DNA, RNA, or a combination thereof.

5. The method of claim 1, wherein incubating the hybridization product in the presence of nucleic acid polymerase produces strand displacement replication of the circular single strand nucleic acids.

6. The method of claim 1, wherein the nucleic acid polymerase has strand displacement activity.

7. The method of claim 1, wherein incubating the hybridization product in the presence of nucleic acid polymerase is performed in the presence of nucleoside triphosphates (NTP) or deoxynucleoside triphosphates (dNTP) comprising a detectable marker.

8. A method of analyzing a target nucleic acid, the method comprising:
amplifying a target nucleic acid according to the method of claim 1; and
measuring the amplified product.

9. A method of amplifying a target nucleic acid, the method comprising:
providing a circular single strand nucleic acid comprising a sequence complementary to at least one strand of a target nucleic acid, and a sample comprising the target nucleic acid;
incubating the circular single strand nucleic acid and the sample to hybridize the circular single strand nucleic acid to the target nucleic acid; and
incubating the hybridization product in the presence of nucleic acid polymerase to amplify the target nucleic acid.

10. The method of claim 9, wherein the circular single strand nucleic acid is DNA, RNA, LNA, or a combination thereof.

11. The method of claim 9, wherein the circular single strand nucleic acid is about 16 nt to about 1,000 nt.

12. The method of claim 9, wherein the target nucleic acid is DNA, RNA, or a combination thereof.

13. The method of claim 9, wherein the incubating of the hybridization product in the presence of nucleic acid polymerase is performed under conditions to catalyze strand displacement replication of the circular single strand nucleic acids.

14. The method of claim 9, wherein the nucleic acid polymerase has strand displacement activity.

15. The method of claim 9, wherein the incubating of the hybridization product in the presence of nucleic acid polymerase is performed in the presence of a second primer comprising a sequence of the circular single strand nucleic acid, wherein the target nucleic acid is single stranded nucleic acid.

16. The method of claim 9, wherein the incubating of the hybridization product in the presence of nucleic acid polymerase is performed in the presence of nucleoside triphosphates (NTP) or deoxynucleoside triphosphates (dNTP) having a detectable marker.

17. A method of analyzing a target nucleic acid in a sample, the method comprising:
amplifying a target nucleic acid according to the method of claim 9; and
measuring the amplified target nucleic acid.

18. A kit to amplify a target nucleic acid, comprising a circular single strand nucleic acids comprising a sequence complementary to a target nucleic acid or comprising a sequence identical to at least a portion of at least one strand of the target nucleic acid; and a nucleic acid polymerase.

19. The kit of claim 18, further comprising a primer having a sequence complementary to the circular single strand nucleic acid or identical to at least a portion of the circular single strand nucleic acid.

20. A composition to amplify target nucleic acids, comprising circular single strand nucleic acids having a sequence complementary to target nucleic acids or having a sequence identical to at least a portion of at least one strand of the target nucleic acids.
